(19) United States
(12) Patent Application Publication
(10) Pub. No.: US 2009/0169487 A1
(43) Pub. Date: Jul. 2, 2009

(54) Essential Oil Diffusion

(76) Inventor: Kamyar Malek HEDAYAT, San Diego, CA (US)

Correspondence Address: AMIN Talati, LLC, 444 NORTH ORLEANS STREET, SUITE 400, CHICAGO, IL, 60654, US

(21) Appl. No.: 12/345,556

(22) Filed: Dec. 29, 2008

Related U.S. Application Data
(60) Provisional application No. 61/017,508, filed on Dec. 28, 2007.

(51) Int. Cl. A61K 9/12; A61K 36/53

(52) U.S. Cl. 424/45; 424/745

(57) ABSTRACT

A method for treating and/or alleviating at least one symptom associated with impaired airway clearance of mucous, impaired oxygen intake, and/or infection in a patient suffering from a respiratory ailment and/or restrictive lung disease includes administering to the patient an effective amount of an essential oil composition including at least one essential oil selected from Lavandula latifolia (spike lavender), Thymus mastichina (Spanish marjoram); Abies balsamea (balsam fir); and Mentha×piperita (peppermint). The symptoms associated with impaired airway clearance, impaired oxygen intake, and/or infection can be alleviated via passive or active inhalation of the essential oil composition.

This application claims the benefit of earlier filed Provisional U.S. Patent Application Ser. No. 61/017,508 filed on Dec. 28, 2007.

### FIELD OF THE INVENTION

The present invention generally relates to a method and formulation for essential oil diffusion for the treatment and/or amelioration of symptoms associated with respiratory conditions. More particularly, the present invention relates to a method and formulation for treating and/or alleviating the symptoms of impaired mucous clearance, impaired oxygen intake, and/or infection such as, for example, Respiratory Syncytial Virus (RSV), bronchiolitis, pneumonia, cystic fibrosis, chronic obstructive pulmonary disease, and other similar conditions.

### BACKGROUND OF THE INVENTION

Children with altered airway clearance represent a small percent of children in the United States. However, such children generally require a disproportionate use of healthcare resources. Additionally, these children suffer an increased degree of morbidity and mortality related to respiratory infections due to their primary disorders, which often results in longer Intensive Care Unit (ICU) stays.

Respiratory Syncytial Virus (RSV) is one of the most frequent infectious agent causing serious respiratory tract infections in children.¹ Approximately 5% of children diagnosed with RSV will require intensive care services.² Additionally, children with certain types of chronic illnesses, such as RSV, impaired airway clearance, cystic fibrosis, and asthma, have a nearly 10 fold increase in likelihood of ICU admission and higher mortality.³ Risk factors for RSV-related hospitalization include prematurity,⁴ congenital heart disease,⁵ and chronic respiratory disorders from anatomical and/or parencymal etiologies.⁶

Various disorders and etiologies can result in impaired airway clearance. For example, scoliosis can lead to bronchial contortion. Muscle weakness from myopathies can result in abnormal cough. Cystic fibrosis can result in altered mucous rheology. Impaired airway clearance can also result in frequent and prolonged primary and secondary infections⁷ and increased oxygen requirement. While more commonly diagnosed in children, these conditions can affect people of all ages.

Currently, the number of commercially available anti-viral agents for respiratory pathogens are limited and have significant costs and side effects. Additionally, current mucolytic treatments have not been demonstrated to be as effective as desired or efficiently delivered to the lungs, particularly in small children.

Known treatments for impaired airway clearance include Dornase Alpha and N-Acetyl Cysteine (NAC) treatment. These treatments have historically had varying rates of success for the treatment of respiratory conditions. Further, lack of efficacy may lead to other complications.

Dornase Alfa (available under the registered trademark “Pulmozyme” from Genentech) is a recombinant human DNase which decreases the viscoelasticity of mucous by cleaving and depolymerizing the DNA in the protein found in sputum.⁸ Dornase Alfa has been used to treat atelectasis from multiple etiologies in the ICU including RSV, bronchiolitis,⁹ and asthma.¹⁰ Adverse effects can include hoarseness, pharyngitis, laryngitis, dyspnea, hemoptysis and bronchospasm.⁹ Dornase Alfa does not appear to be effective in all patients and has been associated with acute onset of desaturation episodes which may be due to altered cough mechanics associated with an underlying myopathy. Further, use of certain mucolytic agents may worsen respiratory dysfunction in such patients.

NAC is the acetylated from of the amino acid cysteine, which alters mucous viscoelasticity by disrupting sulfur bonds of glycoproteins in sputum. Side effects of NAC can include bronchospasm, fever, chills and anaphylaxis.⁹ Neither nebulized nor oral NAC has been found to be of benefit for patients with altered mucous rheology in a systematic review of published studies.¹¹

Thus, in view of the above, there is a need and a demand for a method and/or formulation for treating and/or alleviating the symptoms associated with RSV and/or other disorders or etiologies which result in impaired airway clearance having increased efficacy over traditional pharmaceutical interventions alone. There is a further need and a demand for a method and/or formulation that is suitable for use as a passive inhalant and/or is suitable for use by children. There is a still further need and a demand for a method and/or formulation which is inexpensive to produce and easy to administer.

### SUMMARY OF THE INVENTION

A general object of the invention is to provide a diffused, passively inhaled essential oil composition and method for using the composition.

A more specific object of the invention is to overcome one or more of the problems described above.

The general object of the invention can be obtained, at least in part, via method for treating at least one symptom associated with impaired airway clearance of mucous or impaired oxygen intake, with or without primary, secondary, or opportunistic infection caused by a bacterium or virus, in a patient suffering from a respiratory disorder. The method includes administering to the patient an effective amount of an essential oil composition including at least one essential oil selected from Lavandula latifolia (spike lavender), Thymus mastichina (Spanish marjoram), Abies balsamea (balsam fir), and Mentha×piperita (peppermint). The essential oil(s) can be used alone or combination with one or more essential oil components selected from 1,8 cineole, alpha-pinene, beta-pinene, 1-limonene, beta-caryophyllene, menthol, menthone, and camphor. At least one symptom associated with impaired airway clearance is alleviated via passive inhalation of the essential oil composition.

The invention further comprehends multiple dosing regimens and presentations based on the area of administration (hospital, office, home, recreational setting, etc.) and severity of illness.

The invention still further comprehends passive or active inhalation of an aerosolized essential oil composition, in solid or liquid form, that is diffused into the air or directly into a patient's oral and/or nasal airways.

The invention additionally comprehends passive inhalation of a liquid essential oil composition that is applied to areas around the patient, such as skin, clothing or bedding.

As used herein, the term “essential oil” refers to any concentrated, hydrophobic liquid containing volatile hydrocarbon compounds derived from plants such as aromatic herbs, aromatic plants, or single compounds isolated therefrom.

As used herein the term “drop” refers to a liquid volume equivalent to about 50 microliters (50 μl) to about 80 μl. For example, three (3) drops of a formulation in accordance with the invention has an equivalent liquid volume of between about 150 μl and 240 μl.

Other objects and advantages will be apparent to those skilled in the art from the following detailed description taken in conjunction with the drawings and examples.

### DETAILED DESCRIPTION

The present invention relates to an inhaled essential oil composition for the treatment and/or alleviation of symptoms associated with impaired airway clearance of mucous or impaired oxygen intake, with or without primary, secondary, or opportunistic infection caused by a bacterium or virus, in a patient suffering from a respiratory disorder.

Essential oils may offer novel mucolytic, bronchodilatory, and anti-microbial properties for patients with altered pulmonary mechanics. Essential oils are polychemical organic substances of which 2-4 compounds will usually make up 60-90% of an essential oil's constituents.¹² Generally, the compounds that make up essential oils are terpine hydrocarbons with functional groups such as ethers, phenols, ketones, alcohols and oxides¹³ which interact with various cell receptors.^{14,15,16}

Patients who have altered pulmonary mechanics with compromised mucociliary activity, oxygen requirement, or pulmonary infections of an acute, sub-acute, or chronic nature are often treated with nebulized N-Acetyl Cysteine, Dornase-Alpha and/or chest physiotherapy. In the presence of altered pulmonary mechanics these types of therapies may worsen pulmonary mechanics due to the inability of the patient to expectorate mucous which then becomes further lodged in the bronchial tree. This type of clinical condition can be treated with a diffusion of one or more of the essential oils Lavandula latifolia (spike lavender), Thymus mastichina (Spanish marjoram), Abies balsamea (balsam fir), and Mentha×piperita (peppermint) or a compound isolated therefrom such as camphor, 1,8 cineole (eucalyptol), alpha-pinene, beta-pinene, 1-limonene, beta-caryophyllene, menthol, and/or menthone.

Essential oil of spike lavender (Lavandula latifolia) has been used traditionally as a mucolytic agent,¹⁷ and has expectorant activity,¹⁸ mainly from the presence of eucalyptol (1,8 cineole) and camphor which improve mucous clearance by passive inhalation.¹⁹ Eucalyptol also suppresses arachadonic acid synthesis, leukotriene synthesis and cytokine production.²⁰ In a double-blind randomized control trial, eucalyptol was demonstrated to have steroid-sparing effects in asthmatics.²¹ The whole spike lavender essential oil also possesses anti-inflammatory activity.²² Eucalyptol has been used for over 100 years for its bronchodilatory properties as well.

Spanish marjoram (Thymus mastichina) is a species of Thyme. Its essential oil contains a high amount (40-70%) of eucalyptol.²³ In addition to the effects noted above for eucalyptol, Spanish marjoram has in vitro anti-viral²⁴ and anti-bacterial activity.²⁵

Balsam fir (Abies balsamea) is an evergreen found throughout North America and Europe. The essential oils of coniferous trees are reputed to have strong antimicrobial action, particularly with regard to pulmonary infections of an acute and chronic nature in addition to bronchodilatory properties.²⁶ Balsam Fir was found effective against Staphylococcus aureus.²⁷

The two most common components of peppermint (Mentha piperita) essential oil are a ketone, menthone, and a monoterpene alcohol, menthol. Menthol improves mucociliary clearance, reduces cough,¹⁹ decreases restlessness in children with bronchitis,²⁸ controls excessive cough²⁹ and improves airway caliber in vitro. Menthol also contributes to peppermint's viricidal³⁰ and anti-bacterial activity against pathogenic agents such as herpes simplex virus, Methicillin-resistant staphylococcus aureus (MRSA) and Vancomycin-resistant enterococcus (VRE).³¹ Menthone contributes to peppermint's strong free radical scavenging activity against human monocytes.^{32,33}

Essential oils have demonstrated several interesting properties which may suggest novel treatment approaches to respiratory disease. Treatment with a diffusion of essential oils is believed to decrease oxygen requirement and other symptoms associated with impaired airway clearance.

Essential oils are volatile liquids that readily enter the atmosphere in vapor phase and subsequently the tracheobronchial tree by passive inhalation. They improve mucociliary clearance and volume of sputum expectoration within 30 minutes of use.¹⁹ Most compounds found in essential oils weigh between 126-300 atomic mass units (amu).³⁴ In contrast, NAC has an atomic weight of 163.2 amu, Albuterol 576 amu, and Dornase Alfa 29,249.6 amu.³⁵ Aerosolized delivery of medications larger than 500 amu are highly dependent on method of delivery and patient effort and can be inefficient, particularly in young children and distressed adults.³⁶ Essential oils in contrast are less than 300 amu and are slowly and passively inhaled, a method which is surmised to deliver aerosolized particles more efficiently to the small airways.³⁷ Further, essential oils may have mucolytic, anti-inflammatory, anti-viral and anti-bacterial properties that can be used as a therapy for those with impaired airways.

Essential oils such as those described herein offer a novel and inexpensive treatment option for patients with altered airway clearance or those suffering from pulmonary illnesses of microbial etiologies. In accordance with certain embodiments, the essential oil compositions can be used alone and/or in combination with mucolytic agents such as, for example, Dornase Alpha and/or N-acetyl-cysteine to treat and/or alleviate symptoms associated with impaired airway clearance. In accordance with certain other embodiments, the essential oil compositions can be used alone or as an adjuvant therapy in combination with bronchodilators such as, for example, albuterol, anti-inflammatory agents such as, for example, chromolyn sodium and/or inhaled corticosteroids, and antibiotic preparations.

In accordance with one embodiment, an essential oil formulation can include or consist of one or more essential oils derived from Spanish marjoram, peppermint, spike lavender, balsam fir, and combinations thereof. Suitably, the essential oil formulation can include each oil in a 1:1 ratio. For example, an essential oil formulation can include Spanish marjoram and spike lavender in a 1:1 ratio.

In accordance with another embodiment, an essential oil formulation can include or consist of: an essential oil containing 20% or greater by composition eucalyptol; a coniferous essential oil containing 10% or greater by composition of each of alpha-pinene, beta-pinene, and 1-limonene, and greater than about 0.02% by composition beta-caryophyllene; an essential oil of the Mentha family containing 20% or greater by composition menthol and/or menthone; and/or a camphorous essential oil containing 5% or greater camphor. The eucalyptol-containing essential oil can be derived from Spanish marjoram, spike lavender, Eucalyptus species, Ravinisara camphora, Rosmarinus ssp., Malaleuca ssp., combinations thereof, or the like. The coniferous essential oil can be derived from, but not limited to, Abies balsamea, other Abies ssp, Pinus ssp., Picea ssp, Cupressus ssp., combinations thereof, or the like. The menthol- and menthone-containing essential oil can be derived from peppermint or other suitable species. The camphorous essential oil can be derived from spike lavender, Rosmarinus ssp., Camphora ssp., combinations thereof or the like.

In an alternative embodiment, an essential oil formulation can include one or more of the essential oils of Spanish marjoram, spike lavender, balsam fir and peppermint alone or in combination with one or more of the compounds 1,8 cineole (eucalyptol), alpha-pinene, beta-pinene, 1-limonene, beta-caryophyllene, camphor, menthol, and menthone.

In another embodiment, the essential oil formulation can include or consist of essential oils derived from spike lavender, Spanish marjoram, balsam fir, and peppermint wherein each oil is present in a ratio of about 1:1:1:1. In accordance with a further embodiment, an essential oil formulation can include or consist of: 8 parts Spanish marjoram; 3 parts peppermint; 8 parts spike lavender; and 5 parts balsam fir.

In a still further embodiment, an essential oil formulation can include or consist of: about 25% to about 40% by volume Spanish marjoram; about 25% to about 40% by volume Spike lavender; about 15% to about 30% by volume balsam fir; and about 10% to about 15% by volume peppermint.

In accordance with certain embodiments, the essential oil formulation can further include a carrier matrix. Suitably, the carrier matrix is non-aromatic and can be in the form of a solid, liquid, gel, cream, or other emulsion. For example, the carrier matrix can include or consist of a non-aromatic liquid selected from water, pharmaceutically acceptable solvents (e.g., ethanol), unscented oils, lightly scented oils, and dispersions or emulsions thereof. Alternatively, the carrier matrix can include or consist of a pharmaceutically acceptable coating such as, but not limited to, dipalmitoyl phosphotidylcholine, albumin, and lactose (60:20:20 by weight) or similar embodiment suitable for use with a dry dispersal device or inhaler.

In accordance with one embodiment the carrier matrix can include or consist of a non-aromatic carrier oil. For example, the essential oil formulation can include an unscented massage oil or other unscented or lightly scented, cosmetically acceptable carrier oil including, but not limited to, vegetable oils, grapeseed oil, avocado oil, jojoba oil, wheat germ oil, sunflower oil, or combinations thereof. For example, in an essential oil formulation suitable for use with a nebulizer, water can be used as the carrier matrix. For topical applications, the carrier matrix should be suitable for use on a patient's skin particularly the skin of the chest, back, and neck.

An essential oil composition suitable for application to a surface of a patient's skin, clothing and/or bedding (i.e., a topical composition) can include or consist of about 0.5% to about 10% by volume of the essential oil composition. In accordance with certain embodiments, a topical essential oil composition for the treatment or alleviation of symptoms associated with impaired airway clearance can include about 1% to about 8% by volume of an essential oil composition including or consisting of: about 25% to about 40% by volume Spanish marjoram; about 25% to about 40% by volume spike lavender; about 15% to about 30% by volume balsam fir; and about 10% to about 15% by volume peppermint. The essential oil(s) can be mixed with or dispersed in a carrier matrix such as, for example, a non-aromatic carrier oil (i.e., an unscented or lightly scented oil).

In accordance with certain other embodiments a topical essential oil composition can include or consist of the about 1% to about 8% by volume of an essential oil composition including or consisting of: about 25% to about 40% by volume 1,8 cineole (eucalyptol); about 25% to about 40% by volume camphor; about 15% to about 30% by volume of a combination of alpha-pinene, beta-pinene, 1-limonene, and beta-caryophyllene; and about 10% to about 15% by volume of menthol and/or menthone.

A method of alleviating symptoms associated with impaired airway clearance includes administering to a patient having such symptoms an essential oil composition including or consisting of an essential oil and/or an essential oil component. The essential oil can be selected from Spanish marjoram, peppermint, spike lavender, balsam fir, or combinations thereof. The essential oil component can be selected from the group consisting of 1,8 cineole, camphor, alpha-pinene, beta-pinene, 1-limonene, beta-caryophyllene, menthol menthone, and combinations thereof.

In accordance with certain embodiments, the essential oil composition can be aerosolized in a powdered or liquid form directly into the nasal or oral airways of a patient such as via a nebulizer or metered dose inhaler. Alternatively, the essential oil composition can be administered via a passive inhalation method such as through diffusion of the composition into the surrounding environment via heat, vaporization and/or forced air.

For example, the essential oil composition can be administered as a nebulized composition in an effective amount of about 0.5-5 drops (about 25 μl to about 250 μl) dispersed in about 1 milliliter (ml) to about 5 ml of a suitable carrier matrix such as distilled water. In accordance with certain embodiments, the nebulized composition can be administered about 3-4 times per day until discomfort resolved. The nebulized composition can be administered to a patient via forced air or ultrasonic nebulization.

Alternatively, the essential oil composition can be administered as a powdered formulation including or consisting about 0.5% to about 5% by weight of the essential oil formulation combined with an inhalable solid matrix. Suitably, the powdered formulation is administered using a dry dispersal device which is designed to deliver a metered dose of the essential oil composition.

In accordance with certain other embodiments, about 1-5 drops (about 50 μl to about 400 μl) of the essential oil composition can be mixed with or dispersed in about 1 teaspoon (about 5 milliliters) of a carrier oil such as a non-aromatic massage oil suitable for cosmetic purposes. The essential oil composition may be applied to a surface of a patient's skin, clothing and/or bedding about 3-4 times per day.

### EXAMPLE

Example Formulation: A 14.75 ml diffuser blend is prepared as follows:

TABLE 1

| Essential Oil | mL | Ratio (parts) | % by Volume |
|---|---|---|---|
| Spanish marjoram | 5 | 8 | 33.60 |
| Peppermint | 1.75 | 3 | 11.86 |
| Spike lavender | 5 | 8 | 20.34 |
| Balsam fir | 3 | 5 | 33.90 |
| Total | 14.75 | 24 | 100.00 |

The essential oil formulation can be can be administered such as via nebulization of about 0.5-5 drops (about 25 μl to about 400 μl) of the formulation dispersed in about 1 ml to about 3 ml of a suitable carrier matrix such as distilled water.

### Experimental Results

A 3 year old female, MW, with central core disorder, a static myopathy affecting type I muscle fibers,³⁸ with an 18 day history of oxygen requirement, acute desaturation episodes on high-flow oxygen and persistent left upper lobe atelectasis who experienced a rapid reduction in oxygen requirement was treated with diffused plant essential oils described in TABLE 1, above, after insufficient or worsening response to traditional treatments of N-Acetyl Cysteine (NAC) and Domase Alfa.

She experienced a three day prodrome of upper and lower respiratory symptoms and increasing oxygen requirement treated initially at home. She was admitted to a local hospital 7 days prior to the current admission where she continued to received oxygen and was started on a 7 day antibiotic course despite a positive respiratory direct fluorescent antibody test for RSV. She was discharged home on 3 liters oxygen and persistent left upper lobe atelectasis. She presented to our pediatric intensive care unit (PICU) within 12 hours of discharge with respiratory distress and central cyanosis, pulse oximetry oxygen saturation (spO₂) 83% off oxygen, 88% on 3 liters per minute (LPM) oxygen. Admitting chest roentgram (CXR) revealed an infiltrate of the left upper lobe persistent from her prior admission.

Past medical history was significant for titanium rib insertion for restrictive lung disease, metal rods for thoracic scoliosis, hip repair for hip dislocation, and gastric feeding tube placement. Her home medication regimen included a multivitamin, calcium carbonate, and levalbuterol 1 unit dose nebulized every 8 hours. Lasix had been added during the prior hospitalization. She normally does not require home oxygen, but had been receiving between oxygen for 10 days when she was admitted to our PICU.

MW was placed on 6 liters humidified, high-flow nasal canula oxygen (NC O₂), achieving a spO₂ of 93-98%. Her pulmonary toilet regimen consisted of percussor chest physiotherapy every four hours and postural drainage. On day 4 of admission, day 14 of oxygen requirement, nebulized 20% n-acetyl cysteine (NAC) every eight hours was added. Oxygen requirement began to improve over the next 24 hours, weaning to 2 LPM NC O₂, but returned to 3 LPM when she remained persistently below spO₂ 93%. Nebulized Dornase Alfa 2.5 mg every twelve hours was added on day 7, day 17 of oxygen requirement. Oxygen requirement remained at 3 LPM with frequent desaturations to the mid 80's despite the intensification of mucolytic therapy.

On day 8, the previous treatments were discontinued and the essential oil mixture described in TABLE 1, above, including Lavandula latifolia (spike lavender), Thymus mastichina (Spanish marjoram), Abies balsamea (balsam fir), and Mentha×piperita (peppermint) was nebulized at the parents' request. Three drops were applied to a fibrous filter inserted into the base of a fan diffuser (Fan Fuser, Cinegraph, Inc. Seal Beach, Calif.). The mixture was diffused every six hours into the room and passively inhaled by MW. Oxygen requirement was reduced to 1.5 LPM within 12 hours. The spO₂ increased to 94-97% with abatement of desaturation episodes and both mucolytic treatments were discontinued. MW was weaned to ¼ LPM NC O₂ on the second day of use wand discharged home with an spO₂ of 97%, on day 10 of admission, day 20 of oxygen requirement.

While particular elements, embodiments, and applications of the present invention have been shown and described, it is understood that the invention is not limited thereto because modifications may be made by those skilled in the art, particularly in light of the foregoing teaching. It is therefore contemplated by the appended claims to cover such modifications and incorporate those features which come within the spirit and scope of the invention.
¹Orive F, Flores J, Teresa M, et al. Acute respiratory infection in pediatric intensive care units. A multicenter prospective study. An Esp Pediatr 1998:48:138-42.
²Tsolia M N, Kafetzis D, Danelatou K, et al. Epidemiology of respiratory syncytial virus bronchiolitis in hospitalized infants in Greece. Eur J Epidemiol 2003; 18(1):55-61.
³Purcell K, Fergie J. Driscoll Children's Hospital respiratory syncytial virus database: risk factors, treatment and hospital course in 3308 infants and young children, 1991 to 2002. Pediatr Infect Dis J 2004; 23(5):418-23.
⁴Cunningham C K, McMillan J A, Gross S J. Rehospitalization for respiratory illness in infants of less than 32 weeks' gestation. Pediatrics 1991; 88(3):527-32.
⁵MacDonald N, Hall C, Suffin S. Respiratory syncytial virus infection in children with congenital heart disease. N Engl J Med 1982 (307):397-400.
⁶Groothuis J R, Gutierrez K M, Lauer B A. Respiratory Syncytial virus infection in children with bronchopulmonary dysplasia. Pediatrics 1988; 82(2):199-203.
⁷Siafakas N M, Vermeire P, Pride N B, et al. Optimal assessment and management of chronic obstructive pulmonary disease (COPD). The European Respiratory Society Task Force. Eur Respir J 1995; 8(8):1398-420.
⁸Larrondo J V, Agut M, Calvo-Torras M A. Antimicrobial activity of essences from labiates. Microbios 1995; 82(332):171-2.
⁹Merkus P J, de Hoog M, van Gent R, de Jongste J C. DNase treatement for atelectasis in infants with severe respiratory syncytial virus bronchiolitis. Eur Repir J 2001; 18(4):734-7.
¹⁰Gershan W M, Rusakow L S, Chetty A, Spaingard M L. Resolution of chronic atelectasis in a child with asthma after aerosolized recombinant human DNAse. Pediatr Pulmonol 1994; 18(4):268-9.
¹¹Duijvestijn Y C, Brand P L. Systematic review of N-acetylcysteine in cystic fibrosis. Acta Paediatr 1999; 88(1):3841.
¹²Schnaubelt K. Advanced Aromatherapy: The Science of Essential Oil Therapy. 1^{st} Ed: Healing Arts Press; 1998.
¹³Schnaubelt K. Medical aromatherapy: healing with essential oils. Berkley, Calif.: Frog; 1999.
¹⁴Boskabady M H, Kiani S, Rakhashandah H. Relaxant effects of Rosa damascene on guinea pig tracheal chains and its possible mechanism(s). J Ethnopharmacol 2006; 106(3):377-82.
¹⁵Damiani C E, Moreira C M, Zhang H T, Creazzo T L, Vassallo D V. Effects of eguenol, an essential oil, on the mechanical and electrical activities of cardiac muscle. J Cardiovasc Pharmacol 2004; 44(6):688-95.
¹⁶Madisch A, Heydenreich C J, Wieland V, Hufnagel R, Hotz J. Treatment of functional dyspepsia with a fixed peppermint oil and caraway oil combination preparation as compared to cisapride. A multicenter, reference-controlled double-blind equivalence study. Arzneimittelforschung 1999; 49(11):925-32.
¹⁷Wildwood C. The encyclopedia of aromatherapy. 1^{st} U.S. Ed. Rochester, Vt.: Healing Arts Press; 1996.
¹⁸Charron J M. Use of Lavandula latifolia as an espectorant. J Altern Complement Med 1997: 3(3):211.
¹⁹Hasani A, Pavia D, Toms N, Dilworth P, Agnew J E. Effect of aromatics on lung mucociliary clearance in patients with chronic airways obstruction. J Altern Complement Med 2003; 9(2):243-9.
²⁰Juergens U R, Engelen T, Racke K, Stober M, Gillissen A, Vetter H. Inhibitory activity of 1,8-cineol (eucalyptol) on cytokine production in cultured human lymphocytes and monocytes. Pulm Pharmacol Ther 2004; 17(5):281-7.
²¹Juergens U R, Stober M, Schmidt-Schilling L, Kleuver T, Vetter H. Antiinflammatory effects of euclyptol (1.8-cineole) in bronchial asthma: inhibition of arachidonic acid metabolism in human blood monocytes ex vivo. Eur J Med Res 1998; 3(9):407-12.
²²Shimizu M, Shogawa H, Matsuzawa T, et al. Anti-inflammatory constitutents of topically applied crude drugs. IV. Constituents and anti-inflammatory effect of Paraguayan crude drug “alhucema” (Lavandula latifolia Vill.). Chem Pharm Bull (Tokyo) 1990; 38(8):2283-4.
²³Stewart D. The Chemistry of essential oils made simple. Marble Hill, Mo.: Care Publications; 2005.
²⁴Schnitzler P, Schon K, Reichling J. Antiviral activity of Australian tea tree oil and eucalyptus oil against herpes simplex virus in cell culture. Pharmazie 2001; 56(4):343-7.
²⁵Penalver P, Fluerta B, Broge C, Astoga R, Romero R, Perea A. Antimicrobial activity of five essential oils against origin strains of the Eterobacteriaceae family. Apmis 2005; 113(1):1-6.
²⁶Valnet J, Tisserand R. The practice of aromatherapy: holistic health and the essential oils of flowers and herbs. New York: Destiny Books; 1982.
²⁷Pichette A, Larouche P L, Lebrun M, Legault J. Composition and antibacterial activity of Abies balsamea essential oil. Phytother Res 2006; 20(5):371-3.
²⁸Berger H, Madrieter H, Jarosch E. Effect of Vaporub on the restlessness of children with acute bronchitis. J Int Med Res 1978; 6(6):491-3.
²⁹Morice A H, Marshall A E, Higgins K S, Grattan T J. Effect of inhaled menthol on citric acid induced cough in normal subjects. Thorax 1994; 49(10):1024-6.
³⁰Schuhmacher A, Reichling J, Schnizler P. Virucidal effect of peppermint oil on the enveloped viruses herpes simplex virus type 1 and type 2 in vitro. Phytomedicine 2003; 10(6-7):504-10.
³¹Nelson R R. In-vitro activities of five plant essential oils against methicillin-resistant Staphylococcus aureus and vacomycin-resistant Enterococcus faecium. J Antimicrob Chemother 1997; 40(2):305-6.
³²Juergens U R, Stober M, Better H. The anti-inflammatory activity of L-menthol compared to mint oil in human monocytes in vitro: a novel perspective for its therapeutic use in inflammatory diseases. Eur J Med Res 1998; 3(12):539-45.
³³Mimica-Dukic N, Bozin B, Sokovic M, Mihajlovic B, Matavulj M. Antimicrobial and antioxidant activities of three Mentha species essential oils. Planta Med 2003; 69(5):413-9.
³⁴Stewart D. The Chemistry of essential oils made simple. Marble Hill, Mo.: Care Publications; 2005.
³⁵Budavari S. The Merck index: an encyclopedia of chemicals, drugs, and biological. 11^{th} ed. Rahway, N.J.: Merck; 1989.
³⁶Coleman D M, Kelly H W, McWilliams B C. Determinants of aerosolized albuterol delivery to mechanically ventilated infants. Chest 1996; 109(6):1607-13.
³⁷Agnew J E. Bronchiolar aerosol deposition and clearance. Eur Respir J 1996; 9(6):1118-22.
³⁸Itali T, Amayasu H, Kuribayashi M, et al. Psychological effects of aromatherapy on chronic hemodialysis patients. sychiatry and Clinical Neurosciences 2000; 54(4):393-7.

I claim:

1. A method for treating or alleviating at least one symptom associated with impaired airway clearance in a patient suffering from a respiratory disorder, comprising:
administering to the patient an effective amount of an essential oil composition including:
at least one essential oil selected from the group consisting a eucalyptol-containing essential oil, a coniferous essential oil, a camphorous essential oil, a menthol- and menthone-containing oil; or
at least one oil component selected from the group consisting of 1,8 cineole, camphor, alpha-pinene, beta-pinene, 1-limonene, beta-carophyllene, menthol, menthone, and combinations thereof; or
combinations thereof.
wherein the at least one symptom associated with impaired airway clearance is alleviated via passive or active inhalation of the essential oil composition.

2. The method according to claim 1, wherein
the eucalyptol-containing essential oil is derived from Spanish marjoram, spike lavender, Eucalyptus species, Ravinisara camphora, Rosmarinus ssp., Malaleuca ssp., or combinations thereof;
the coniferous essential oil is derived from Abies balsamea, other Abies ssp, Pinus ssp., Picea ssp, Cupressus ssp., or combinations thereof;
the camphorous essential oil is derived from spike lavender, Rosmarinus ssp., Camphora ssp., or combinations thereof.
the menthol- and menthone-containing essential oil is derived from peppermint, other Mentha species, or combinations thereof.

3. The essential oil composition according to claim 2, wherein:
the eucalyptol-containing essential oil comprises at least about 20% eucalyptol;
the coniferous essential oil comprises at least about 10% alpha-pinene, at least about 10% beta-pinene, at least about 10% 1-limonene, and at least about 0.02% beta-caropyllene;
the camphorous essential oil comprises at least about 5% camphour; and
the menthol- and menthone-containing essential oil comprises at least about 20% menthol and at least about 20% menthone.

4. The method according to claim 1, wherein essential oil composition comprises:
about 25% to about 40% by volume spike lavender;
about 25% to about 40% by volume Spanish marjoram;
about 15% to about 30% balsam fir; and
about 10% to about 25% peppermint.

5. The method according to claim 1, wherein the effective amount of the essential oil comprises about 25 microliters (μl) to about 400 μl.

6. The method according to claim 1, wherein the administering step further comprises diffusing the essential oil composition into the patient's surrounding environment.

7. The method according to claim 6, wherein in the essential oil composition is diffused into the surrounding environment via at least one of heat, vaporization, forced air ventilation, or a combinations thereof.

8. The method according to claim 1, wherein the essential oil composition further comprises a carrier matrix.

9. The method according to claim 8, wherein the essential oil composition is administered via direct aerosolization into the patient's airways via ultrasonic nebulization, a metered dose inhaler, or a combination thereof.

10. The method according to claim 9, wherein the effective amount of the essential oil composition comprises about 25 μl to about 250 μl of the essential oils dispersed in about 1 milliliter (ml) to about 5 ml of the carrier matrix.

11. The method according to claim 8, wherein the administering step further comprises applying the essential oil composition onto a surface of the patient's skin, clothing, bedding, or a combinations thereof.

12. A method for treating or alleviating at least one symptom associated with impaired airway clearance in a patient suffering from a respiratory disorder, comprising:
administering to the patient an effective amount of an essential oil composition including:
about 25% to about 40% by volume spike lavender;
about 25% to about 40% by volume Spanish marjoram;
about 15% to about 30% by volume balsam Fir;
about 10% to about 25% by volume peppermint; and
optionally, a carrier matrix;
wherein the at least one symptom associated with impaired airway clearance is alleviated via inhalation of the essential oil composition.

13. The method according to claim 12, wherein the carrier matrix comprises a non-aromatic liquid selected from the group consisting of water, pharmaceutically acceptable solvents, unscented oils, lightly scented oils, and dispersions or emulsions thereof, or a pharmaceutically acceptable solid inhalant.

14. The method according to claim 12, wherein the essential oil composition is administered via aerosolization of the composition and inhalation is via a passive mechanism, an active mechanism or a combination thereof.

15. The method according to claim 12, wherein the administering step further comprises diffusing the essential oil composition into the patient's surrounding environment via at least one of heat, vaporization, forced air ventilation, or a combination thereof.

16 The method according to claim 14, wherein in the essential oil composition is diffused directly into the patient's nasal and/or oral passages via ultrasonic nebulization, metered dose inhaler, or a combination thereof.

17. The method according to claim 12, wherein the essential oil composition comprises a topical composition including about 0.5% to about 10% by volume of the essential oil composition dispersed in a carrier matrix including a non-aromatic carrier oil selected from the group consisting of vegetable oils, grapeseed oil, avocado oil, jojoba oil, wheat germ oil, sunflower oil, and combinations thereof.

18. The method according to claim 17, wherein the administering step further comprises applying the essential oil composition onto a surface of the patient's skin, clothing, bedding, or a combinations thereof.

19. A method for the treatment of at least one symptom associated with impaired airway clearance in a patient suffering from a respiratory disorder, comprising:
administering to the patient an essential oil composition including about 8 parts spike lavender, about 8 parts Spanish marjoram, about 5 parts balsam fir, about 3 parts peppermint;
wherein the essential oil composition is administered in one or more doses, a dose including between 25 μl and 400 μl of the essential oil composition.

20. The method according to claim 19, wherein the doses are administered in time periods of one dose per minute up to, and including, one dose per week and the doses are administered at a hospital, an office, a home, or a recreational setting.
